(19) United States
(12) Patent Application Publication
(10) Pub. No.: US 2009/0120808 A1
(43) Pub. Date: May 14, 2009

(54) Device for Detecting Methanol Concentration and the Method Thereof

(75) Inventors: Hung-Chang Wu, Taichung County (TW); Jui-Lung Chien, Taipei County (TW); Chien-Hsiao Chen, Taipei County (TW); Hsiu-Ping Pearl Lin, Taipei (TW)

Correspondence Address: BAKER & MCKENZIE LLP, Pennzoil Place, South Tower, 711 Louisiana, Suite 3400, HOUSTON, TX, 77002-2716, US

(73) Assignee: Development Center for Biotechnology, Taipei County (TW)

(21) Appl. No.: 11/932,650

(22) Filed: Oct. 31, 2007

(30) Foreign Application Priority Data
Jun. 20, 2007 (TW) 096122119

(51) Int. Cl. G01N 27/327; C12Q 1/32; G01N 27/26

(52) U.S. Cl. 205/777.5; 435/26; 204/403.14

(57) ABSTRACT

A device for detecting methanol concentration in an alcohol-containing solution is disclosed. The device implements an electrochemical bio-detector based on a two-enzyme system to quickly, easily and accurately measure methanol concentration in an alcohol-containing solution at a relatively low cost. A method for detecting methanol concentration in a sample using the same device is also disclosed.

### CROSS-REFERENCE TO RELATED APPLICATIONS

This application claims priority to Taiwan Patent Application No. 096122119 filed on Jun. 20, 2007.

### FIELD OF THE INVENTION

The invention relates to a device for detecting methanol concentration in an alcohol-containing solution and the detection method thereof. In particular, the present invention relates to a method and a device for measuring the methanol concentration in an alcohol-containing solution by using a two-enzyme system.

### BACKGROUND OF THE INVENTION

Methanol intoxication usually occurs when a user consumes alcoholic drinks that contain high concentration of methanol, and in severe cases can result in blindness or even death. Methanol, often referred to as “wood alcohol,” is often used as a solvent in a variety of industries. Methanol is also used as a fuel, as a paint remover or as a denaturing reagent. A small trace of methanol may be found in some alcoholic drinks.

The metabolism of methanol is mainly carried out in the liver, where an enzyme called alcohol dehydrogenase oxidizes the methanol to formaldehyde and formic acid. Formaldehyde and formic acid are roughly 33 and 6 times more toxic than methanol, respectively. The major symptoms of acute methanol intoxication are: (i) depression of the central nervous system; (ii) accumulation of formic acid; and (iii) visual degeneration. When suffering methanol intoxication, a large amount of ethanol may be administered to the patient because ethanol competitively binds to alcohol dehydrogenase and prevents the metabolism of methanol, so that less formaldehyde and formic acid will be produced. Therefore, ethanol can be used to treat methanol intoxication.

Currently there are several ways to measure the amount of methanol in an alcohol-containing solution, including chromatography and gas chromatography. The Chromotropic Acid Test is one of the most popular tests, a standard published by the Association of Official Agricultural Chemists. Such tests, however, has some drawbacks. First, a good quantitative result will only be obtained when all reaction conditions are under strict control, and the test result may be positively biased if carbohydrate exists in the tested sample. Furthermore, for samples tested by this method, several pre-treating steps are required, and it takes more than 4 hours to analyze one sample. In addition, the reagents used in this method are highly toxic and have negative impacts on health and the environment, making this method less suitable for the general public.

Although gas chromatography can provide accurate measurement, it is still not desirable due to the long pre-treating and analyzing steps, high cost, large volume of the instruments, and the requirement of highly trained personnel.

Biosensors are detectors that comprise a bio-detecting element and a signal transmitting element. Derived from traditional enzyme electrodes, biosensors involve bio-catalytic and bio-affinity capabilities. Moreover, biosensors have the following advantages that overcome the above-mentioned drawbacks of the traditional detecting method: (1) biosensors can be repeatedly used by applying the fixation techniques to fix the reagents; (2) biosensors have high specificity and can significantly lower the background noise; (3) biosensors typically have simple structures thus making them easy to use; (4) the sensitivity is high and therefore only a small amount of sample is required; (5) biosensors have short response time and a result can be obtained quickly; and (6) biosensors can have digital signal output and therefore the physical dimensions can be minimized for portable purposes and on-site detection.

In view of the above, it is desirable to provide a micro-biosensor that is portable, user-friendly, cost-effective and can quickly measure methanol concentration to prevent methanol intoxication.

The methanol/ethanol rapid detector currently available on the market is also a biosensor. It utilizes an alcohol oxidase (AOX) to oxidize methanol/ethanol and to produce H₂O₂, followed by application of voltage (or combining with peroxidase) to oxidize H₂O₂ and release electrons. The concentration of methanol/ethanol in the solution can then be determined by the so-measured current. This mechanism has one major drawback that the alcohol oxidase will oxidize both methanol and ethanol, and therefore the so-measured current does not distinguish methanol from ethanol.

NADH is a compound of high reducing power that exists in cells. NADH functions mainly as a coenzyme in cells to provide hydrogen atoms necessary in enzyme reaction and is oxidized to NAD+. Generally a redox enzyme needs NADH as a coenzyme to facilitate the reaction. Pseudomonas putida glutathione-independent formaldehyde dehydrogenase (FDH) has the advantage where it can directly convert formaldehyde and NAD+ to formic acid and NADH without involving glutathione as a coenzyme. NADH can be widely applied to industrial and commercial use due to its strong reducing power. Taiwan patent application No. 096116235 describes a cost-effective, genetically mutated FDH whose amino acid sequence has been changed to improve the enzyme activity and substrate specificity.

The present invention combines the molecular biology techniques with an electrochemical enzymatic bio-detector to provide a bio-detector that can detect the concentration of methanol/formaldehyde in a liquid or alcohol sample. Methanol intoxication can be prevented by using the bio-detector of the present invention to determine if methanol is present in alcoholic drinks.

### SUMMARY OF THE INVENTION

An aspect of the present invention is to provide a method for measuring the methanol concentration in an alcohol-containing solution, comprising the steps of: (1) oxidizing the methanol in the solution to formaldehyde with an Alcohol Oxidase (“AOX”); (2) oxidizing, in the presence of NAD+, the formaldehyde to formic acid with a Formaldehyde Dehydrogenase (“FDH”) while reducing the NAD+ to NADH; (3) reacting the NADH with an electron mediator to oxidize the NADH to NAD+; (4) generating an oxidation current by having the electron mediator releases electrons after auto-oxidation; and (5) measuring the value of the oxidation current and plugging the value in a pre-established linear equation for methanol concentration and current value to determine methanol concentration in the solution.

Another aspect of the present invention is to provide a methanol detecting device for the detection of methanol concentration in an alcohol-containing solution, comprising: a substrate having a reference electrode, a working electrode and an active area provided thereon, the reference electrode, the working electrode and the active area being separated from each other, the working electrode having a working area comprising an AOX, a FDH, and an electron mediator.

Still another aspect of the present invention is to provide a method for measuring the methanol concentration in a sample by using a methanol detecting device comprising a substrate having a reference electrode and a working electrode provided thereon, the working electrode being separate from the reference electrode and having a working area comprises an AOX, a FDH, and an electron mediator, the working electrode and the reference electrode each connected to a corresponding terminal of a potentiostat, the method comprising: (1) measuring an initial current value (Iᵢ) by contacting the methanol-detecting device with an initial electrolyte solution; (2) adding a predetermined amount of the sample to the initial electrolyte solution and, in the presence of NAD+, measuring a final current value (I_{f}); (3) calculating a current difference (ΔI) between the initial current value and the final current value (Iᵢ−I_{f}); and (4) determining the methanol concentration in the sample by substituting the current difference (ΔI) in a pre-established linear equation for the current difference and methanol concentration.

By combining molecular biology, enzyme fixation and electrochemical-relating techniques, the present invention provides a genetically-modified FDH having improved specificity to formaldehyde in a bio-detector to quickly and accurately detect methanol concentration. The bio-detector is easy to use, cost-effective, and can provide real-time result. The methanol detecting device of the present invention can be directly used in measuring the methanol concentration in alcoholic drinks to prevent methanol intoxication.

### BRIEF DESCRIPTION OF THE DRAWINGS

FIG. 1 is a reaction scheme illustrating the method of the present invention to measure the methanol concentration.

FIG. 2 is a schematic view illustrating the structure of the SPE electrode of the present invention.

FIG. 3A is a graph showing the current output obtained using an SPE electrode that was prepared without RS to measure a solution of constant concentration of methanol.

FIG. 3B is a graph showing the current output obtained by using an SPE electrode that was prepared with RS to measure a solution of constant concentration of methanol.

FIGS. 4A-4C are graphs showing the current output obtained by using SPE electrodes prepared by different MB-RS to Carbon gel ratio to detect the methanol concentration.

FIG. 5 is a graph showing the current output obtained by using SPE electrodes prepared by different AOX to FDH ratio to detect the methanol concentration under the same amount of NAD+.

FIG. 6 is a graph showing the relationship between the current difference and methanol concentration obtained by adding different amount of NAD+ to the tested sample.

FIG. 7A is a graph showing the relationship between the current difference and NADH concentration using “batch-detecting” method.

FIG. 7B is a graph showing the relationship between the current difference and NADH concentration using “continuous-detecting” method.

FIG. 8 is a graph showing the relationship between the current difference and methanol concentration using three SPE electrodes manufactured in three different batches.

FIG. 9 is a graph showing the relationship between the current difference and methanol concentration when the operating voltage is optimized to 400 mV.

FIG. 10 is a graph showing the relationship between the current difference and a broad range of ethanol concentration.

FIG. 11 is a graph showing the relationship between the current difference and methanol concentration under constant ethanol concentration.

FIG. 12 is a flowchart illustrating the method for measuring methanol concentration in an alcohol-containing solution according to the present invention.

### DESCRIPTION OF EMBODIMENTS OF THE INVENTION

In the prior art methanol concentration is measured by first oxidizing methanol with an AOX to produce formaldehyde and hydrogen peroxide, followed by oxidizing the hydrogen peroxide with external voltage to release electrons, and measuring the electrical current to determine the methanol concentration. Such method has a major drawback in that an AOX catalyzes not only methanol but also ethanol. Therefore from the resulting current it is impossible to distinguish methanol from ethanol, making it difficult to determine methanol concentration. The present invention, however, describes a bio-detecting method with a dual-enzyme system that combines an AOX with a FDH. By measuring the oxidation current generated by the reaction between an electron mediator and NADH, the methanol concentration can be determined accordingly.

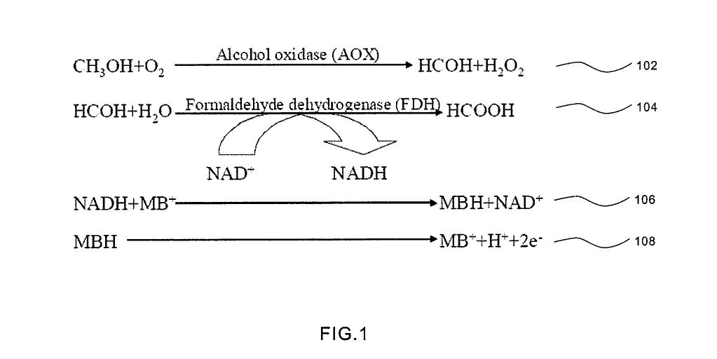

A dual-enzyme system combining AOX with FDH is implemented in the present invention as a bio-sensor to replace the single AOX system in the prior art. Please refer to FIG. 1, which illustrates the method for measuring methanol concentration in an alcohol-containing solution. In step 102 the methanol in the solution is oxidized with an AOX to give formaldehyde and hydrogen peroxide. In step 104, in the presence of NAD+ the formaldehyde is oxidized to formic acid with a FDH while NAD+ is reduced to NADH. In step 106, an electron mediator such as Meldola Blue (“MB”) reacts with NADH to oxidize NADH to NAD+. At the same time, in step 108 the electron mediator releases electrons to form an oxidation current by auto-oxidation. Finally, the oxidation current is measured and the measured value is plugged into a pre-established linear equation for methanol concentration and current value to determine methanol concentration.

The above-mentioned electron mediator is not limited as long as it can oxidize NADH. Non-limiting examples include Meldola Blue (MB, 8-dimethylamino-2,3-benzophenoxazine), Prussian Blue (potassium hexacyanoferrate), dichlorophenolindophenol, p-benzoquinone, o-phenylenediamine and 3,4-dihydroxybenzaldehyde. Preferably, Meldola Blue is implemented as the electron mediator in the present invention.

The above-mentioned “pre-established linear equation for methanol concentration and current value” is a standard reference equation for methanol concentration and current value. The equation is established by measuring solutions of known methanol concentration with the method of the present invention to obtain the corresponding current values of those methanol concentrations.

The following examples are designed to verify the accuracy of the method according to the present invention. Example 1 describes the preparation of a screen printing electrode (SPE) and enzyme fixation. Example 2 illustrates the high solubility of MB and the relationship between different MB-RS ratio and the current output signals. In Example 3 the optimal enzyme ratio is determined. In Example 4 the standard curves between NADH concentration and current value are shown to confirm that the method of the present invention is valid. In Example 5 the relationship between methanol concentration and current output signal is established. In Example 6 it is shown that a noise signal of ethanol can be deducted. In Example 7 the procedure of measuring methanol concentration is described based on the above Examples.

Unless defined otherwise, all the technical and scientific terms described herein are as commonly known to those skilled in the art. Note that persons skilled in the art can also understand other methods and materials similar to the present invention that can be used to practice the present invention.

### Example 1

### The Preparation of SPE and Enzyme Fixation

The materials suitable for the electrode of the present invention include but not limited to indium oxide, glass, gold, platinum, palladium, graphite and carbon black. The structure of the electrode is not limited as long as it can be used to practice the present invention without adverse effect. Suitable structures of the electrode include planar electrode, hollow needle electrode and solid needle electrode. The surface of the electrode is preferably cleaned by acids, bases, physical polishing or ultrasound treatment. In this example a screen printing electrode (SPE) is described.

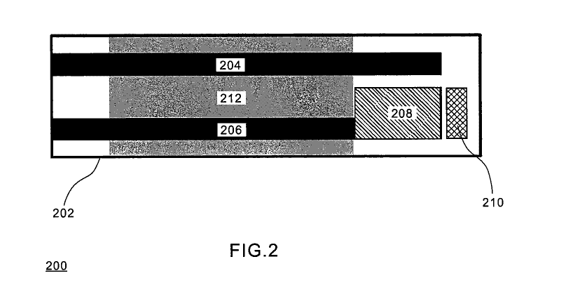

The SPE used in this example was bought from TaiDoc Technology Corporation and has a structure shown in FIG. 2. SPE 200 includes a substrate 202 made of PVC, a reference electrode 204 provided on the substrate 202, a working electrode 206 provided on the substrate 202 and separated from the reference electrode 204, and an active region 210 provided on the substrate 202 and separated from the reference and working electrodes 204, 206, wherein the working electrode 206 includes a working region 208 comprising AOX, FDH and an electron mediator, and the active region 210 comprises NAD+. On top of these electrodes and active region there is provided a blue insulating layer 212 made of PVC. As shown in FIG. 2, the area of working region 208 is intentionally enlarged such that the amount of enzymes and electron mediator contained therein can be increased to enhance current output signal and to lower the minimum of detecting range.

To facilitate enzyme fixation, SPE 200 is first processed with a sonicator for 20 minutes. AOX, FDH and an electron mediator, which is MB in this example, are then fixed to the working region 208 of the SPE 200 via the following steps: MB is electrically polymerized on the working region 208 under CV mode; a mixture of AOX/FDH with certain ratio of activity unit is mixed with a 10% gelatin solution, and a certain amount of the mixed solution is added dropwise to the working region 208; 2.5% of glutaraldehyde is added to the working region 208 for cross-linking; and after air-dried the electrode is used for testing. During the preparation process, NAD+ is separately fixed to an active region 210 on the substrate 202 instead of being fixed to the working region 208 along with AOX, FDH and the electron mediator. The reason being that NAD+ can be easily reduced to NADH if it is co-fixed with AOX, FDH and electron mediator, which will adversely affect the shelf life, stability and reproducibility of the electrode.

It should be noted that although NAD+ is separately provided on the electrode in this embodiment, it is not the only configuration. For example, NAD+ can be added to the sample solution through other ways, such as adding a tablet containing NAD+ to the sample solution, instead of being directly fixed to the SPE of the methanol detection device.

### Example 2

### High Solubility of Mb and the Relationship Between MB/RS Ratio and the Current Output Signal

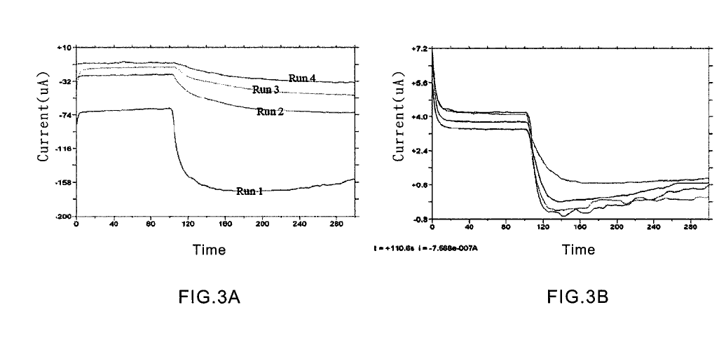

FIG. 3A shows the result of using the SPE prepared from Example 1 to continuously measure the methanol concentration of a testing solution in which the methanol concentration is constant. FIG. 3A shows that as the cycle count increases the oxidation current signal becomes less sharp and less consistent. The color of testing solution in testing vial changed from transparent to blue, indicating that MB was dissolved in the solution. This result suggests that MB is highly soluble, and can significantly affect the stability of the electrode.

To overcome the problem of high solubility of MB, 0.1 M of MB was first mixed with 0.1 M of Reinecke salt to form a precipitate of MB-RS complex. After collecting the MB-RS complex by centrifugation and dried for 30 minutes, the resulting product was grounded to powder and mixed with carbon gel in a predetermined ratio. The resulting gel was directly coated to the working region 208. Thereafter, Example 1 was repeated to fix AOX and FDH on the resulting working region 208 prepared above. FIG. 3B shows a current output signal using this SPE to carry out multiple methanol measurement. It is shown that the use of MB-RS complex makes the current signal much more constant as compared to FIG. 3A. During the measuring process the color of the testing solution changed only mildly, indicating that the problem of high solubility of MB has been resolved.

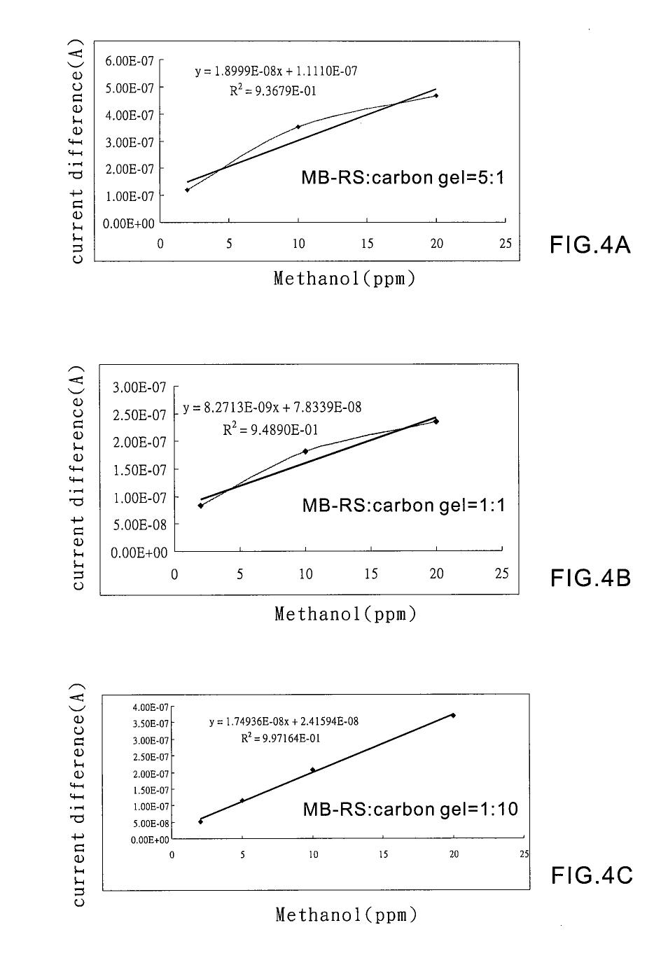

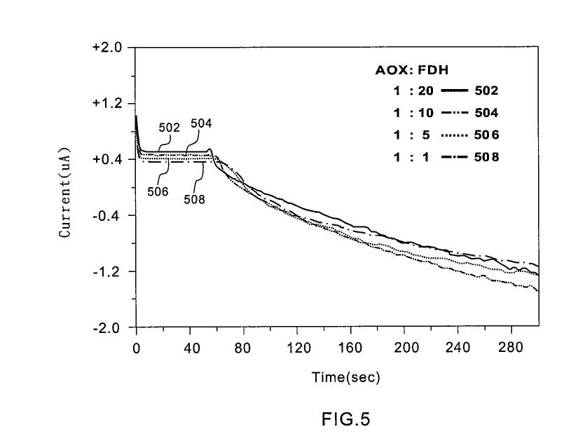

To further increase the stability of the SPE, the inventors of the present invention explores the possibility of mixing carbon gel into the working region 208. FIGS. 4A to 4C show the relationship between current difference and methanol concentration, obtained by using a SPE 200 having different ratio of MB-RS to carbon gel in the working region 208 to measure methanol concentration. The term “current difference” refers to the difference between the initial value (the plateau) and final value (after the sharp drop) of the current output signal shown in FIG. 3B. In FIG. 4A the ratio of MB-RS to carbon gel is 5:1. In FIG. 4B the ratio of MB-RS to carbon gel is 1:1. In FIG. 4C the ratio of MB-RS to carbon gel is 1:10. FIGS. 4A to 4C show that the relationship between the current difference and methanol concentration is linear when the ratio of MB-RS to carbon gel is within 5:1 to 1:10. More preferably, the ratio of MB-RS to carbon gel is 1:10 to provide better SPE performance.

### Example 3

### Optimal Enzyme Ratio

In this example electrodes made of different AFX/FDH ratio were used to measure methanol concentration under the same NAD+ condition. The current output signals are as shown in FIG. 5. Current output signal 502 represents the volume ratio of AOX to FDH being 1:20; current output signal 504 represents the volume ratio of AOX to FDH being 1:10; current output signal 506 represents the volume ratio of AOX to FDH being 1:5; and current output signal 502 represents the volume ratio of AOX to FDH being 1:1. In this example 1.3 mg of NAD+ was added, and the enzyme activity per milliliter of AOX and FDH is 1020 U/ml and 550 U/ml, respectively. Table 1 lists the ratio of enzyme activity with corresponding volume ratio between AOX and FDH. When the AOX:FDH activity ratio falls within the range of 1 U:0.5-11 U the effect of the present invention can be successfully carried out. Note that even under the activity ratio of AOX:FDH as 1 U:0.5 U, FDH is still in excess amount, and therefore the range of activity ratio can be further broadened. Theoretically an activity ratio of 10.1 to 1:20 of AOX:FDH should be reasonable.

TABLE 1

| Volume ratio of AOX:FDH | Activity ratio of AOX:FDH |
|---|---|
| 1:20 | 1.02 U:11 U |
| 1:10 | 1.02 U:5.5 U |
| 1:5 | 1.02 U:2.75 U |
| 1:1 | 1.02 U:0.55 U |

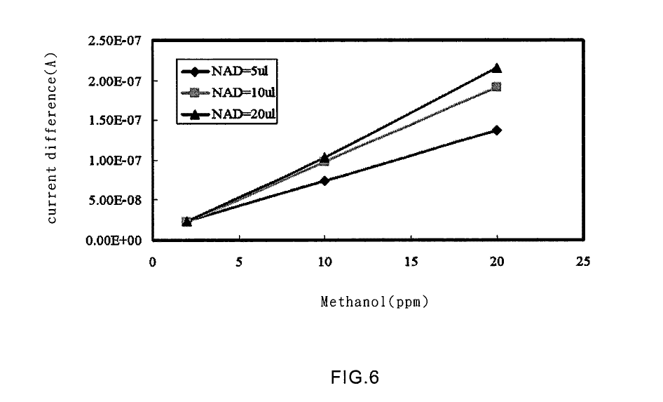

FIG. 6 illustrates the result of adding different volume of NAD+ in the testing solution. FIG. 6 shows that the amount of NAD+ in the testing solution does not change the linear correlation between the current difference and methanol concentration, indicating that in the dual-enzyme system of the present invention the concentration of NAD+ is independent from the testing result.

### Example 4

### Standard Curve of NADH Concentration

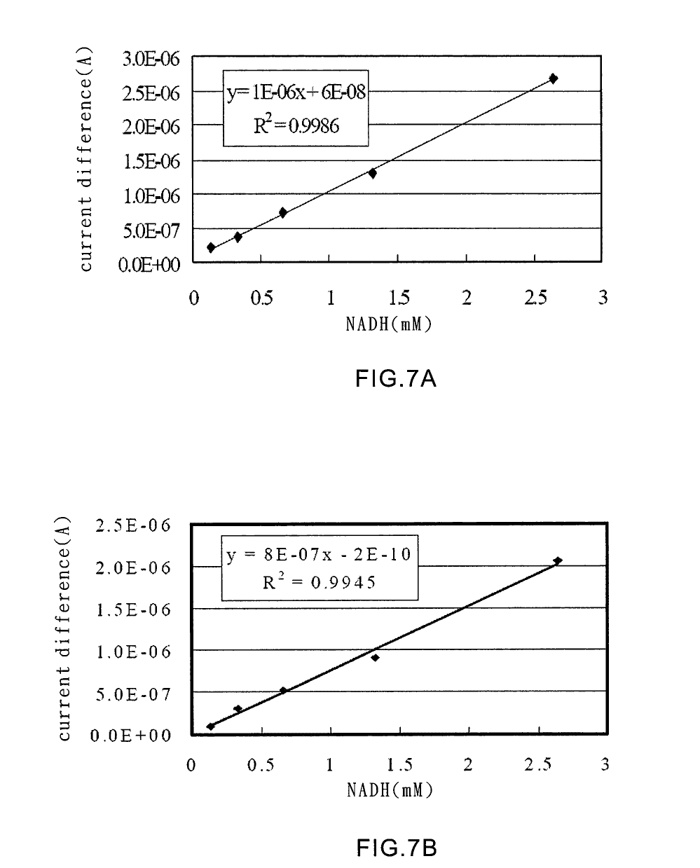

The purpose of this example is to ensure that a current difference can be obtained through the oxidation current generated by the method of the present invention. The correlation between the current difference and NADH concentration is shown in FIGS. 7A and 7B by first using the MB-RS SPE 200 prepared in Example 2 to measure a current output signal and then correlating the current difference with NADH concentration. In the experiment of FIG. 7A the result was obtained through a “batch-measuring” fashion, whereas in the experiment of FIG. 7B a continuous-measuring fashion was implemented. “Batch-measuring” refers to a protocol where after a set of data is obtained all the solution in the testing vials is discarded and a new batch of solution is made, and the electrode is then washed by deionized water and ready for the next set of experiment. “Continuous-measuring” refers to a protocol where once the current output signal of the previous experiment becomes constant, the same solution and electrode are used to carry out the next set of experiment by adding different amount of NADH, and through this protocol the reusability of the electrode can be tested. For batch-measuring, each data was obtained by taking the average of at least three sets of independent experiments. For continuous measuring, the data was obtained from one single experiment. FIGS. 7A and 7B show that the current different increases as NDAH concentration increases, regardless of using either batch-measuring or continuous measuring. Such result indicates that NADH does react with the Meldola Blue in the working region 208 of the SPE 200, and electrons are released to working electrode 206 to generate an oxidation current, which is translated into the current difference.

### Example 5

### Methanol Concentration and Current Output Signal

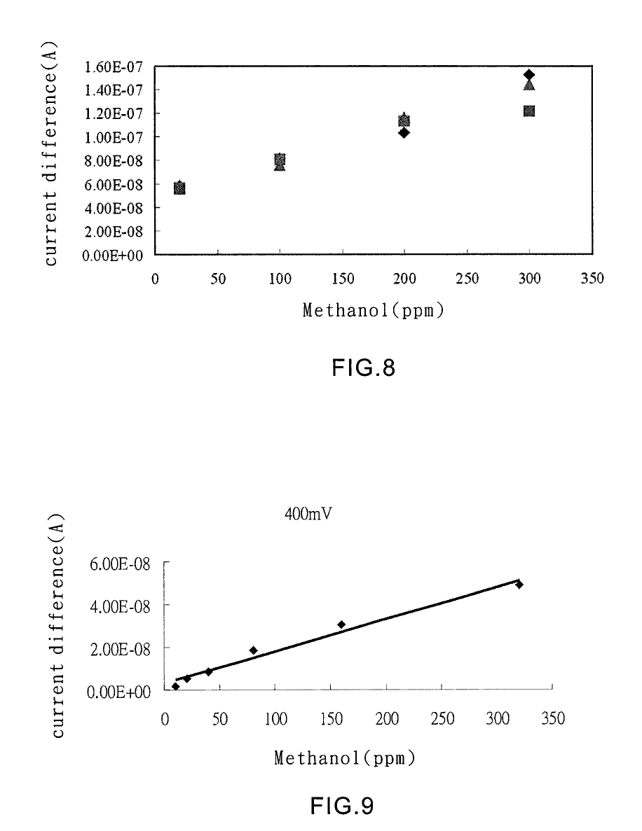

FIG. 8 is a graph showing the correlation between methanol concentration and current difference, in which the data was obtained by using SPE 200 prepared in three different batches. FIG. 8 shows that when the methanol concentration falls in the range between 0 to 300 mg/L, the current difference linearly correlates with the methanol concentration regardless of the batch number of SPE 200. The SPE 200 manufactured in different batches shows rather similar result.

FIG. 9 shows that when the operation voltage is 400 mV, the current difference linearly correlates with methanol concentration in the range of 0 to 325 mg/L. In the present invention the current difference linearly correlates with methanol concentration in the range of approximately 0 to 300 mg/L. The linear equation that describes the relationship between current difference and methanol concentration can be expressed as follows: ΔI=mx+B, where ΔI is the current difference, x is the methanol concentration, and m & B are the constants that can be derived from the experimental data in FIG. 9.

### Example 6

### Deduction of Noise Signal from Ethanol

When methanol and ethanol have the same concentration, such as 20 mg/L, the current difference for methanol is 3.0×10-7 A, for ethanol is 2×10-8 A. In other words, the current output signal for methanol is a dozen times higher than that for ethanol, meaning the FDH of the present invention has very high specificity to formaldehyde.

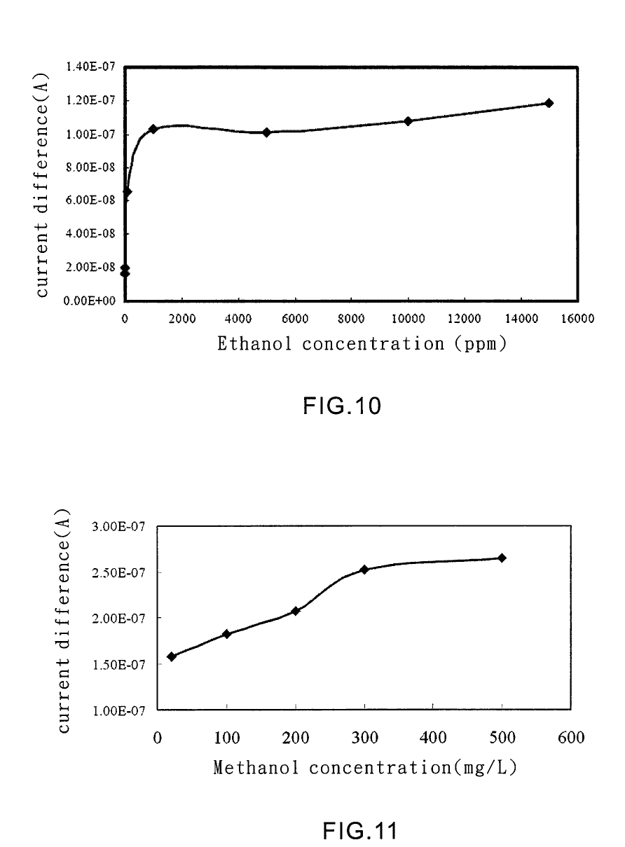

FIG. 10 shows the relationship between current difference and a broad range of ethanol concentration. This experiment is conducted to verify the influence of ethanol to the method described by the present invention. FIG. 10 shows that the current output signal increases rapidly as the ethanol concentration increases. Once the ethanol concentration reaches 1000 mg/L the current difference tapers down and becomes almost constant. The physical meaning of this constant is that though the FDH of the present invention is highly specific to formaldehyde, given that the ethanol concentration in liquor is much higher than methanol, the acetaldehyde converted from ethanol by AOX will still provide a certain amount of background noise. FIG. 10 also shows that once the ethanol concentration is higher than 1,000 mg/L the current difference becomes constant. Since normally the concentration of ethanol in liquors is at least three to four orders greater than 1,000 mg/L, the current difference of ethanol can be deducted as a constant.

FIG. 11 is a graph showing the relationship between current difference and different methanol concentration in the same amount of ethanol. FIG. 11 shows that when methanol concentration is in the range of 0 to 300 mg/L the current difference linearly increases as methanol concentration increases. This suggests that the existence of ethanol will not affect the current output signal, hence the current difference, of methanol. In other words, when measuring the methanol concentration the current difference of ethanol can be deducted first.

### Example 7

### Procedures of Measuring Methanol Concentration

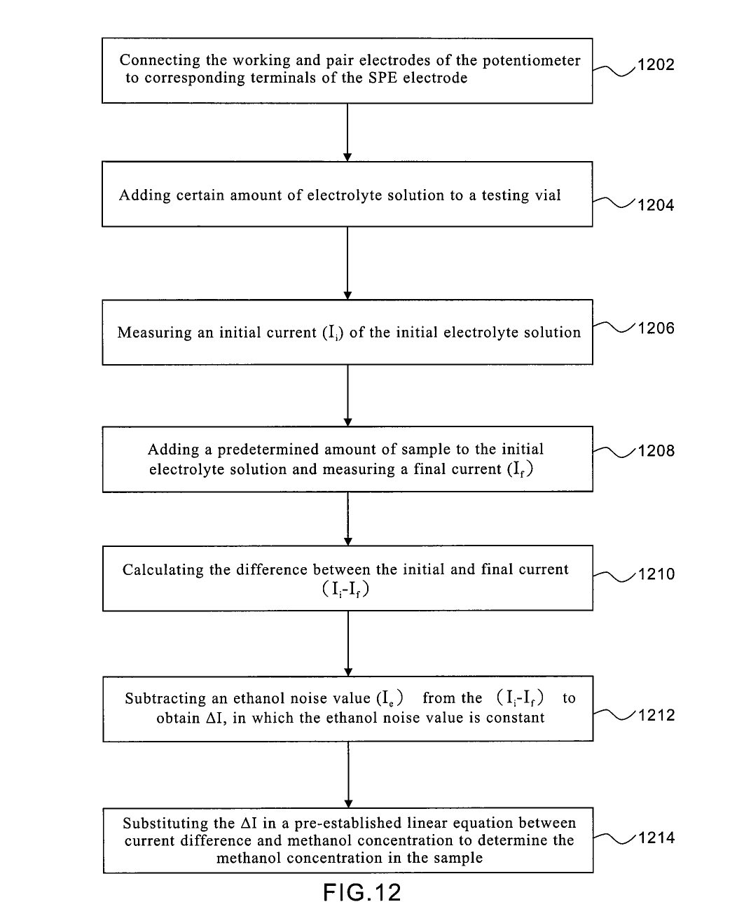

FIG. 12 is a flowchart showing the steps for measuring methanol concentration. In step 1202 the SPE 200 from Example 2 is placed in a testing vial, and the terminals of the potentiometer are connected to corresponding terminals on the working electrode and pair electrode of SPE 200. In step 1204a predetermined amount of electrolyte, such as a PBS-KCl buffer, is added to the testing vial to pre-treat the SPE 200. In step 1206 an initial current output, Iᵢ, is measured. In step 1208a predetermined amount of sample is added to the electrolyte and a final current output, I_{f}, is measured. In step 1210a difference of current output (Iᵢ−I_{f}) is calculated by subtracting the final current output I_{f} from the initial current output Iᵢ. In step 1212a ΔI is obtained by subtracting an ethanol noise signal I_{c} from the (Iᵢ−I_{f}), wherein the ethanol noise signal Iₑ is a constant such as that shown in FIG. 11. In step 1214 the ΔI is plugged into a pre-established linear equation for methanol concentration and current difference to determine the methanol concentration in the sample.

The “pre-established linear equation for methanol concentration and current difference” mentioned above is established by adding different samples of known methanol concentration and then correlating the measured current difference with those known methanol concentration, as illustrated in FIG. 9. The equation can be expressed in the form of: ΔI=mx+B where ΔI is the current difference, x is the methanol concentration, and m & B are constants derived from the experimental data.

Although ethanol signal Iₑ has already been subtracted from the above-mentioned current difference (ΔI), it is not a necessary step. As shown in FIG. 11, the ethanol noise signal will not affect the current output of methanol. Given that the ethanol noise signal remains nearly constant once the ethanol concentration reaches beyond 1,000 mg/L, adding the ethanol noise signal to the linear equation will lead to the same result. In other words, an alternative current difference ΔI′=Iᵢ−I_{f} can be used instead of ΔI=Iᵢ−I_{f}−Iₑ, to be plugged into the linear equation, and the correlation between this alternative current difference ΔI′ and methanol concentration can still be made with the same accuracy. Another way to establish the linear equation is to use a solution of high ethanol concentration as a solvent to which methanol is added, and the linear equation for the current difference (ΔI′=Iᵢ−I_{f}) and methanol concentration will by itself include the ethanol noise.

While the present invention is disclosed by reference to the preferred embodiments and examples detailed herein, it is to be understood that these examples are intended in an illustrative rather than in a limiting sense. It is contemplated that modifications and combinations will readily occur to those skilled in the art, which modifications and combinations will be within the spirit of the invention and the scope of the following claims.

What is claimed is:

1. A method for measuring methanol concentration in an alcohol-containing solution, comprising:
a) oxidizing the methanol in the solution to formaldehyde with an Alcohol Oxidase (AOX);
b) oxidizing, in the presence of NAD+, said formaldehyde to formic acid with a
Formaldehyde Dehydrogenase (FDH) while reducing said NAD+ to NADH;
c) reacting said NADH with an electron mediator to oxidize said NADH to NAD+
d) generating an oxidation current by having said electron mediator release electrons after auto-oxidation; and
e) measuring the value of said oxidation current and plugging said value in a pre-established linear equation for methanol concentration and current value to determine methanol concentration in said solution.

2. The method of claim 1, wherein said electron mediator is selected from a group consisting of: Meldola Blue (MB, 8-dimethylamino-2,3-benzophenoxazine), Prussian Blue (potassium hexacyanoferrate), dichlorophenolindophenol, p-benzoquinone, o-phenylenediamine, 3,4-dihydroxybenzaldehyde and the mixture thereof.

3. The method of claim 1, wherein the enzyme activity ratio between said AOX and said FDH ranges from 1:0.1 to 1:20.

4. A methanol detecting device for the detection of methanol concentration in an alcohol-containing solution, comprising:
a substrate having a reference electrode and a working electrode provided thereon, said working electrode being separate from said reference electrode and having a working area comprising an Alcohol Oxidase (AOX), a Formaldehyde Dehydrogenase (FDH), and an electron mediator.

5. The methanol detecting device of claim 4, wherein said electron mediator is selected from a group consisting of: Meldola Blue (MB, 8-dimethylamino-2,3-benzophenoxazine), Prussian Blue (potassium hexacyanoferrate), dichlorophenolindophenol, p-benzoquinone, o-phenylenediamine, 3,4-dihydroxybenzaldehyde and the mixture thereof.

6. The methanol detecting device of claim 5, wherein said working area of said working electrode further comprises Reinecke salt.

7. The methanol detecting device of claim 6, wherein said electron mediator in said working area is Meldola Blue that forms a complex compound with said Reinecke salt, and the weight ratio between said Meldola Blue and said Reinecke salt is approximately 1:1.

8. The methanol detecting device of claim 7, wherein said working area of said working electrode further comprises carbon gel.

9. The methanol detecting device of claim 8, wherein the weight ratio between said carbon gel and said complex compound ranges from 1:0.2 to 1:10.

10. The methanol detecting device of claim 4, wherein said device further comprises an active region provided on said substrate and separated from said reference electrode and said working electrode, wherein said active region comprises NAD+.

11. The methanol detecting device of claim 10, wherein said active region is provided in proximity to said working area of said working electrode.

12. The methanol detecting device of claim 4, wherein the enzyme activity ratio between said AOX and said FDH ranges from 1:0.1 to 1:20.

13. A method for measuring methanol concentration in a sample by using a methanol detecting device comprising a substrate having a reference electrode and a working electrode provided thereon, said working electrode being separated from said reference electrode and having a working area comprising an Alcohol Oxidase (AOX), a Formaldehyde Dehydrogenase (FDH), and an electron mediator, said working electrode and said reference electrode each connected to a corresponding terminal of a potentiostat, the method comprising:
a) measuring an initial current value (Iᵢ) by contacting said methanol-detecting device with an initial electrolyte solution;
b) adding a predetermined amount of said sample to said initial electrolyte solution and, measuring a final current value (I_{f}) in the presence of NAD+;
c) calculating a current difference (ΔI) between said initial current value and said final current value (Iᵢ−I_{f}); and
d) determining methanol concentration in said sample by plugging said current difference (ΔI) in a pre-established linear equation for current difference and methanol concentration.

14. The method of claim 13, wherein said methanol detecting device further comprises an active region provided on said substrate and separated from said reference electrode and working electrode, said active region comprising NAD+.

15. The method of claim 13, wherein said pre-established linear equation is established by using said methanol detecting device to detect different solutions of known methanol concentration.

16. The method of claim 13, wherein said pre-established linear equation is valid when the methanol concentration ranges from 0 to 300 mg/L.

17. The method of claim 13, wherein said step c) further comprises: calculating a current difference (ΔI) by subtracting said final current value and a noise value of ethanol (Iₑ) from said initial current value (Iᵢ−I_{f}−Iₑ).
